(19) United States
(12) Patent Application Publication
(10) Pub. No.: US 2008/0033006 A1
(43) Pub. Date: Feb. 7, 2008

(54) 1,2,4-Triazolo&lsqb; 1,5-A&rsqb; Pyridines as Gastric Acid Secretion Inhibitors

(75) Inventor: Wilm Buhr, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 10/586,613

(22) PCT Filed: Jan. 25, 2005
(86) PCT No.: PCT/EP05/50300

§ 371 Date: Jul. 19, 2006

(30) Foreign Application Priority Data
Jan. 26, 2004 (EP) 04001563.8

(51) Int. Cl. A61K 31/437; A61P 1/00; C07D 471/04

(52) U.S. Cl. 514/303; 546/119

(57) ABSTRACT

The invention provides compounds of the formula 1,

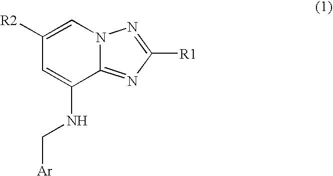

in which the substituents and symbols are defined in the description. The compounds inhibit the secretion of gastric acid.

### TECHNICAL FIELD

The invention relates to novel compounds which are used in the pharmaceutical industry as active compounds for preparing medicaments.

### PRIOR ART

U.S. Pat. No. 4,358,454 describes differently substituted 1,2,4-triazolo[1,5-a]pyridines, which compounds are said to be useful in the treatment of peptide ulcer.

The International Patent Application WO 02/048145 describes 1,2,4-triazolo[1,5-a]pyridine derivatives which have a good affinity to the adenosine receptor and may therefore be used in the treatment of diseases related to this receptor.

The International Patent Application WO 01/17999 and WO 02/048145 describe aminotriazolopyridine derivatives which compounds are said to be adensine receptor ligands.

The International Patent Applications WO 99/55705, WO 99/55706, WO 00/11000, WO 03/018582 describe substituted imdazo pyridine derivatives, which compounds inhibit gastric acid secretion.

### DESCRIPTION OF THE INVENTION

The invention provides compounds of the formula I

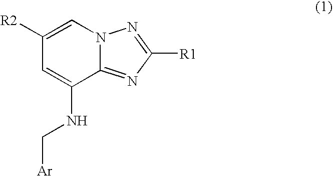

where
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is halogen, fluoro-1-4C-alkyl, 2-4C-alkenyl, 2-4C-alkynyl, carboxyl, cyano, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, amino-1-4C-alkyl, mono- or di-1-4C-alkylamino-1-4C-alkyl, the radical Res-1-4C-alkyl or the radical —CO—NR31R32,
where
   Res is a imidazo, morpholino, aziridino, azetidino, pyrrolidino, pyrrolo, piperidino, piperazino or a with R30 substituted benzylamino radical and the radical Res is bonded via its nitrogen atom or one of its nitrogen atoms to the 1-4C-alkyl radical
   where
      R30 is 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen or hydroxy,
   R31 is hydrogen, hydroxyl, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
or where
   R31 and R32 together, including the nitrogen atom to which both are bonded, are a pyrrolidino, piperidino, piperazino, N-1-4C-alkylpiperazino, morpholino, aziridino or azetidino group,
Ar is one with R4, R5, R6 and R7 substituted mono- or bicyclic aromatic residue from the group of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
wherein
   R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, fluoro-1-4C-alkyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R5 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, fluoro-1-4C-alkyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl
   R6 is hydrogen, 1-4C-alkyl or halogen and
   R7 is hydrogen, 1-4C-alkyl or halogen,
wherein
   aryl is phenyl or substituted phenyl with one, two or three same or different substituents from the group of 1-4C-alkyl, 1-4C-alkoxy, carboxy, 1-4C-alkoxycarbonyl, halogen, trifluoromethyl, nitro, trifluoromethoxy, hydroxy and cyano.
with the proviso that R1 does not have the meaning hydrogen, 1-4C-alkyl or hydroxy-1-4C-alkyl when R2 has the meaning halogen or fluoro-1-4C-alkyl,
   and the salts of these compounds.

1-4C-Alkyl denotes straight-chain or branched alkyl radicals having 1 to 4 carbon atoms. Examples which may be mentioned are the butyl, isobutyl, sec-butyl, tert-butyl, propyl, isopropyl, ethyl and methyl radicals.

3-7C-Cycloalkyl denotes cyclopropyl, cyclobutyl, cyclopentyl, cyclohexyl and cycloheptyl, among which cyclopropyl, cyclobutyl and cyclopentyl are preferred.

3-7C-Cycloalkyl-1-4C-alkyl denotes one of the abovementioned 1-4C-alkyl radicals which is substituted by one of the abovementioned 3-7C-cycloalkyl radicals. Examples which may be mentioned are the cyclopropylmethyl, the cyclohexylmethyl and the cyclohexylethyl radicals.

1-4C-Alkoxy denotes radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy and preferably the ethoxy and methoxy radicals.

1-4C-Alkoxy-1-4C-alkyl denotes one of the abovementioned 1-4C-alkyl radicals which is substituted by one of the abovementioned 1-4C-alkoxy radicals. Examples which may be mentioned are the methoxymethyl, the methoxyethyl and the butoxyethyl radicals.

1-4C-Alkoxycarbonyl (—CO-1-4C-alkoxy) denotes a carbonyl group to which is attached one of the abovementioned 1-4C-alkoxy radicals. Examples which may be mentioned are the methoxycarbonyl (CH₃O—C(O)—) and the ethoxycarbonyl (CH₃CH₂O—C(O)—) radicals.

2-4C-Alkenyl denotes straight-chain or branched alkenyl radicals having 2 to 4 carbon atoms. Examples which may be mentioned are the 2-butenyl, 3-butenyl, 1-propenyl and the 2-propenyl (allyl) radicals.

2-4C-Alkynyl denotes straight-chain or branched alkynyl radicals having 2 to 4 carbon atoms. Examples which may be mentioned are the 2-butynyl, the 3-butynyl and, preferably, the 2-propynyl (propargyl radicals).

Fluoro-1-4C-alkyl denotes one of the abovementioned 1-4C-alkyl radicals which is substituted by one or more fluorine atoms. An example which may be mentioned is the trifluoromethyl radical.

Hydroxy-1-4C-alkyl denotes one of the abovementioned 1-4C-alkyl radicals which is substituted by a hydroxyl group. Examples which may be mentioned are the hydroxymethyl, the 2-hydroxyethyl and the 3-hydroxypropyl radicals.

For the purpose of the invention, halogen is bromine, chlorine and fluorine.

1-4C-Alkoxy-1-4C-alkoxy denotes one of the abovementioned 1-4C-alkoxy radicals which is substituted by a further 1-4C-alkoxy radical. Examples which may be mentioned are the radicals 2-(methoxy)ethoxy (CH₃—O—CH₂—CH₂—O—) and 2-(ethoxy)ethoxy (CH₃—CH₂—O—CH₂—CH₂—O—).

1-4C-Alkoxy-1-4C-alkoxy-1-4C-alkyl denotes one of the abovementioned 1-4C-alkoxy-1-4C-alkyl radicals which is substituted by one of the abovementioned 1-4C-alkoxy radicals. An example which may be mentioned is the radical 2-(methoxy)ethoxymethyl (CH₃—O—CH₂—CH₂—O—CH₂—).

Fluoro-1-4C-alkoxy-1-4C-alkyl denotes one of the abovementioned 1-4C-alkyl radicals which is substituted by a fluoro-1-4C-alkoxy radical. Here, fluoro-1-4C-alkoxy denotes one of the abovementioned 1-4C-alkoxy radicals which is fully or predominantly substituted by fluorine. Examples of fully or predominantly fluorine-substituted 1-4C-alkoxy which may be mentioned are the 1,1,1,3,3,3-hexafluoro-2-propoxy, the 2-trifluoromethyl-2-propoxy, the 1,1,1-trifluoro-2-propoxy, the perfluoro-tert-butoxy, the 2,2,3,3,4,4,4-heptafluoro-1-butoxy, the 4,4,4-trifluoro-1-butoxy, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy radicals.

Amino-1-4C-alkyl denotes one of the abovementioned 1-4C-alkyl radicals which are substituted by an amino group. Examples which may be mentioned are the aminomethyl, the 2-aminoethyl and the 3-aminopropyl radicals.

Mono- or di-1-4C-alkylamino radicals contain, in addition to the nitrogen atom, one or two of the abovementioned 1-4C-alkyl radicals. Preference is given to di-1 -4C-alkylamino and in particular to dimethyl-, diethyl- or diisopropylamino.

Mono- or di-1-4C-alkylamino-1-4C-alkyl denotes one of the abovementioned 1-4C-alkyl radicals which is substituted by one of the abovementioned mono- or di-1-4C-alkylamino radicals. Examples which may be mentioned are the dimethylaminomethyl, the dimethylaminoethyl and the diethylaminomethyl radicals.

1-7C-Alkyl denotes straight-chain or branched alkyl radicals having 1 to 7 carbon atoms. Examples which may be mentioned are the heptyl, isoheptyl-(5-methylhexyl), hexyl, isohexyl-(4-methylpentyl), neohexyl-(3,3-dimethylbutyl), pentyl, isopentyl-(3-methylbutyl), neopentyl-(2,2-dimethylpropyl), butyl, isobutyl, sec-butyl, tert-butyl, propyl, isopropyl, ethyl and methyl radicals.

1-4C-Alkylcarbonyl denotes a radical which, in addition to the carbonyl group, contains one of the abovementioned 1-4C-alkyl radicals. An example which may be mentioned is the acetyl radical.

2-4C-Alkenyloxy denotes a radical which, in addition to the oxygen atom, contains a 2-4C-alkenyl radical. An example which may be mentioned is the allyloxy radical.

Carboxy-1-4C-alkyl denotes, for example, the carboxymethyl (—CH₂COOH) or the carboxyethyl (—CH₂CH₂COOH) radical.

1-4C-Alkoxycarbonyl-1-4C-alkyl denotes one of the abovementioned 1-4C-alkyl radicals which is substituted by one of the abovementioned 1-4C-alkoxycarbonyl radicals. An example which may be mentioned is the ethoxycarbonylmethyl (CH₃CH₂OC(O)CH₂—) radical.

Aryl-1-4C-alkyl denotes an aryl-substituted 1-4C-alkyl radical. An example which may be mentioned is the benzyl radical.

Aryl-1-4C-alkoxy denotes an aryl-substituted 1-4C-alkoxy radical. An example which may be mentioned is the benzyloxy radical.

1-4C-Alkylcarbonylamino denotes an amino group to which a 1-4C-alkylcarbonyl radical is attached. Examples which may be mentioned are the propionylamino (C₃H₇C(O)NH—) and the acetylamino (acetamido, CH₃C(O)NH—) radicals.

1-4C-Alkoxycarbonylamino denotes an amino radical which is substituted by one of the abovementioned 1-4C-alkoxycarbonyl radicals. Examples which may be mentioned are the ethoxycarbonylamino and the methoxycarbonylamino radicals.

1-4C-Alkoxy-1-4C-alkoxycarbonyl denotes a carbonyl group to which one of the abovementioned 1-4C-alkoxy-1-4C-alkoxy radicals is attached. Examples which may be mentioned are the 2-(methoxy)-ethoxycarbonyl (CH₃—O—CH₂CH₂—O—CO—) and the 2-(ethoxy)ethoxycarbonyl (CH₃CH₂—O—CH₂CH₂—O—CO—) radicals.

1-4C-Alkoxy-1-4C-alkoxycarbonylamino denotes an amino radical which is substituted by one of the abovementioned 1-4C-alkoxy-1-4C-alkoxycarbonyl radicals. Examples which may be mentioned are the 2-(methoxy)ethoxycarbonylamino and the 2-(ethoxy)ethoxycarbonylamino radicals.

Radicals Ar which may be mentioned are, for example, the following substituents: 4-acetoxyphenyl, 4-acetamidophenyl, 2-methoxyphenyl, 3-methoxyphenyl, 4-methoxyphenyl, 3-benzyloxyphenyl, 4-benzyloxyphenyl, 3-benzyloxy-4-methoxyphenyl, 4-benzyloxy-3-methoxyphenyl, 3,5-bis(trifluoromethyl)phenyl, 4-butoxyphenyl, 2-chlorophenyl, 3-chlorophenyl, 4-chlorophenyl, 2-chloro-6-fluorophenyl, 3-chloro-4-fluorophenyl, 2-chloro-5-nitrophenyl, 4-chloro-3-nitrophenyl, 3-(4-chlorophenoxy)phenyl, 2,4-dichlorophenyl, 3,4-difluorophenyl, 2,4-dihydroxyphenyl, 2,6-dimethoxyphenyl, 3,4-dimethoxy-5-hydroxyphenyl, 2,5-dimethylphenyl, 3-ethoxy-4-hydroxyphenyl, 2-fluorophenyl, 4-fluorophenyl, 4-hydroxyphenyl, 2-hydroxy-5-nitrophenyl, 3-methoxy-2-nitrophenyl, 3-nitrophenyl, 2,3,5-trichlorophenyl, 2,4,6-trihydroxyphenyl, 2,3,4-trimethoxyphenyl, 2-hydroxy-1-naphthyl, 2-methoxy-1-naphthyl, 4-methoxy-1-naphthyl, 1-methyl-2-pyrrolyl, 2-pyrrolyl, 3-methyl-2-pyrrolyl, 3,4-dimethyl-2-pyrrolyl, 4-(2-methoxycarbonylethyl)-3-methyl-2-pyrrolyl, 5-ethoxycarbonyl-2,4-dimethyl-3-pyrrolyl, 3,4-dibromo-5-methyl-2-pyrrolyl, 2,5-dimethyl-1-phenyl-3-pyrrolyl, 5-carboxy-3-ethyl-4-methyl-2-pyrrolyl, 3,5-dimethyl-2-pyrrolyl, 2,5-dimethyl-1-(4-trifluoromethylphenyl)-3-pyrrolyl, 1-(2,6-dichloro-4-trifluoromethylphenyl)-2-pyrrolyl, 1-(2-nitrobenzyl)-2-pyrrolyl, 1-(2-fluorophenyl)-2-pyrrolyl, 1-(4-trifluoromethoxyphenyl)-2-pyrrolyl, 1-(2-nitrobenzyl)-2-pyrrolyl, 1-(4-ethoxycarbonyl)-2,5-dimethyl-3-pyrrolyl, 5-chloro-1,3-dimethyl-4-pyrazolyl, 5-chloro-1-methyl-3-trifluoromethyl-4-pyrazolyl, 1-(4-chlorobenzyl)-5-pyrazolyl, 1,3-dimethyl-5-(4-chlorophenoxy)-4-pyrazolyl, 1-methyl-3-trifluoromethyl-5-(3-trifluoromethylphenoxy)-4-pyrazolyl, 4-methoxycarbonyl-1-(2,6-dichlorophenyl)-5-pyrazolyl, 5-allyloxy-1-methyl-3-trifluoromethyl-4-pyrazolyl, 5-chloro-1-phenyl-3-trifluoromethyl-4-pyrazolyl, 3,5-dimethyl-1-phenyl-4-imidazolyl, 4-bromo-1-methyl-5-imidazolyl, 2-butylimidazolyl, 1-phenyl-1,2,3-triazol-4-yl, 3-indolyl, 4-indolyl, 7-indolyl, 5-methoxy-3-indolyl, 5-benzyloxy-3-indolyl, 1-benzyl-3-indolyl, 2-(4-chlorophenyl)-3-indolyl, 7-benzyloxy-3-indolyl, 6-benzyloxy-3-indolyl, 2-methyl-5-nitro-3-indolyl, 4,5,6,7-tetrafluoro-3-indolyl, 1-(3,5-difluorobenzyl)-3-indolyl, 1-methyl-2-(4-trifluorophenoxy)-3-indolyl, 1-methyl-2-benzimidazolyl, 5-nitro-2-furyl, 5-hydroxymethyl-2-furyl, 2-furyl, 3-furyl, 5-(2-nitro-4-trifluoromethylphenyl)-2-furyl, 4-ethoxycarbonyl-5-methyl-2-furyl, 5-(2-trifluoromethoxyphenyl)-2-furyl, 5-(4-methoxy-2-nitrophenyl)-2-furyl, 4-bromo-2-furyl, 5-dimethylamino-2-furyl, 5-bromo-2-furyl, 5-sulfo-2-furyl, 2-benzofuryl, 2-thienyl, 3-thienyl, 3-methyl-2-thienyl, 4-bromo-2-thienyl, 5-bromo-2-thienyl, 5-nitro-2-thienyl, 5-methyl-2-thienyl, 5-(4-methoxyphenyl)-2-thienyl, 4-methyl-2-thienyl, 3-phenoxy-2-thienyl, 5-carboxy-2-thienyl, 2,5-dichloro-3-thienyl, 3-methoxy-2-thienyl, 2-benzothienyl, 3-methyl-2-benzothienyl, 2-bromo-5-chloro-3-benzothienyl, 2-thiazolyl, 2-amino-4-chloro-5-thiazolyl, 2,4-dichloro-5-thiazolyl, 2-diethylamino-5-thiazolyl, 3-methyl-4-nitro-5-isoxazolyl, 2-pyridyl, 3-pyridyl, 4-pyridyl, 6-methyl-2-pyridyl, 3-hydroxy-5-hydroxymethyl-2-methyl-4-pyridyl, 2,6-dichloro-4-pyridyl, 3-chloro-5-trifluoromethyl-2-pyridyl, 4,6-dimethyl-2-pyridyl, 4-(4-chlorophenyl)-3-pyridyl, 2-chloro-5-methoxycarbonyl-6-methyl-4-phenyl-3-pyridyl, 2-chloro-3-pyridyl, 6-(3-trifluoromethylphenoxy)-3-pyridyl, 2-(4-chlorophenoxy)-3-pyridyl, 2,4-dimethoxy-5-pyrimidine, 2-quinolinyl, 3-quinolinyl, 4-quinolinyl, 2-chloro-3-quinolinyl, 2-chloro-6-methoxy-3-quinolinyl, 8-hydroxy-2-quinolinyl and 4-isoquinolinyl.

Suitable salts of compounds of the formula 1 are—depending on the substitution—in particular all acid addition salts. Particular mention may be made of the pharmacologically acceptable salts of the inorganic and organic acids customarily used in pharmacy. Those suitable are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, where the acids are employed in the salt preparation in an equimolar ratio or in a ratio differing therefrom, depending on whether the acid is a mono- or polybasic acid and on which salt is desired.

Pharmacologically unacceptable salts, which can be initially obtained, for example, as process products in the preparation of the compounds according to the invention on an industrial scale, are converted into pharmacologically acceptable salts by processes known to the person skilled in the art.

It is known to the person skilled in the art that the compounds according to the invention and their salts can, for example when they are isolated in crystalline form, comprise varying amounts of solvents. The invention therefore also embraces all solvates and, in particular, all hydrates of the compounds of the formula 1, and all solvates and, in particular, all hydrates of the salts of the compounds of the formula 1.

Compounds which are to be emphasized are those of the formula 1,

where
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is halogen, fluoro-1-4C-alkyl, 2-4C-alkenyl, 2-4C-alkynyl, carboxyl, cyano, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, amino-1-4C-alkyl, mono- or di-1-4C-alkylamino-1-4C-alkyl, the radical Res-1-4C-alkyl or the radical —CO—NR31R32,
where
   Res is a imidazo, morpholino, aziridino, azetidino, pyrrolidino, pyrrolo, piperidino, piperazino or a with R30 substituted benzylamino radical and the radical Res is bonded via its nitrogen atom or one of its nitrogen atoms to the 1-4C-alkyl radical
   where
      R30 is 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen or hydroxy,
   R31 is hydrogen, hydroxyl, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
or where
   R31 and R32 together, including the nitrogen atom to which both are bonded, are a pyrrolidino, piperidino, piperazino, N-1-4C-alkylpiperazino, morpholino, aziridino or azetidino group,
Ar is one with R4, R5, R6 and R7 substituted mono- or bicyclic aromatic residue from the group of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
wherein
   R4 is hydrogen, 1-4C-alkyl, hydroxy-1-4C-alkyl, 1-4C-alkoxy, halogen or fluoro-1-4C alkyl
   R5 is hydrogen, 1-4C-alkyl, hydroxy-1-4C-alkyl, 1-4C-alkoxy, halogen or fluoro-1-4C alkyl
   R6 is hydrogen,
   R7 is hydrogen,
with the proviso that R1 does not have the meaning hydrogen, 1-4C-alkyl or hydroxy-1-4C-alkyl when R2 has the meaning halogen or fluoro-1-4C-alkyl,
   and the salts of these compounds.

Particular mention may be made of those compounds of the formula 1,

where
R1 1-4C-alkyl, 3-7C-cycloalkyl, 3-7C-cycloalkyl-1-4C-alkyl, 1-4C-alkoxy-1-4C-alkyl, fluoro-1-4C-alkyl, hydroxy-1-4C alkyl,
R2 is carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, amino-1-4C-alkyl, mono- or di-1-4C-alkylamino-1-4C-alkyl, the radical Res-1-4C-alkyl or the radical —CO—NR31R32,
where
   Res is a imidazo, morpholino, aziridino, azetidino, pyrrolidino, pyrrolo, piperidino or piperazino radical and the radical Res is bonded via its nitrogen atom or one of its nitrogen atoms to the 1-4C-alkyl radical
   R31 is hydrogen, 1-7C-alkyl, hydroxy-1-4C-alkyl or 1-4C-alkoxy-1-4C-alkyl,
   R32 is hydrogen, 1-7C-alkyl or 1-4C-alkoxy-1-4C-alkyl,
Ar is one with R4, R5, R6 and R7 substituted monocyclic aromatic residue selected from the group of phenyl, pyridinyl, thiophenyl, furanyl and pyrrolyl,
wherein
   R4 is hydrogen, 1-4C-alkyl, halogen or fluoro-1-4C-alkyl,
   R5 is hydrogen, 1-4C-alkyl, halogen or fluoro-1-4C-alkyl,
   R6 is hydrogen
   R7 is hydrogen
      and the salts of these compounds.

Particular mention may also be made of those compounds of the formula 1,

where
R1 1-4C-alkyl, 3-7C-cycloalkyl, 3-7C-cycloalkyl-1-4C-alkyl, 1-4C-alkoxy-1-4C-alkyl, fluoro-1-4C-alkyl, hydroxy-1-4C alkyl,
R2 is carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, amino-1-4C-alkyl, mono- or di-1-4C-alkylamino-1-4C-alkyl, the radical Res-1-4C-alkyl or the radical —CO—NR31R32,
where
   Res is a imidazo, morpholino, aziridino, azetidino, pyrrolidino, pyrrolo, piperidino or piperazino radical and the radical Res is bonded via its nitrogen atom or one of its nitrogen atoms to the 1-4C-alkyl radical
   R31 is hydrogen, 1-7C-alkyl, hydroxy-1-4C-alkyl or 1-4C-alkoxy-1-4C-alkyl,
   R32 is hydrogen, 1-7C-alkyl or 1-4C-alkoxy-1-4C-alkyl,
Ar is one with R4, R5, R6 and R7 substituted phenyl,
wherein
   R4 is hydrogen or 1-4C-alkyl
   R5 is hydrogen or 1-4C-alkyl
   R6 is hydrogen
   R7 is hydrogen
      and the salts of these compounds.

Particular emphasis is given to compounds of the formula 1,

where
R1 1-4C-alkyl
R2 is carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, amino-1-4C-alkyl, mono- or di-1-4C-alkylamino-1-4C-alkyl, the radical Res-1-4C-alkyl or the radical —CO—NR31 R32,
where
   Res is a imidazo or morpholino radical and is bonded via its nitrogen atom or one of its nitrogen atoms to the 1-4C-alkyl radical
   R31 is 1-4C-alkyl or 1-4C-alkoxy-1-4C-alkyl,
   R32 is hydrogen, 1-4C-alkyl or 1-4C-alkoxy-1-4C-alkyl,
Ar is one with R4, R5, R6 and R7 substituted phenyl,
wherein
   R4 is hydrogen or 1-4C-alkyl
   R5 is hydrogen or 1-4C-alkyl
   R6 is hydrogen
   R7 is hydrogen
      and the salts of these compounds.

Among the compounds of the formula 1, those compounds of the formula 1-a are preferred.

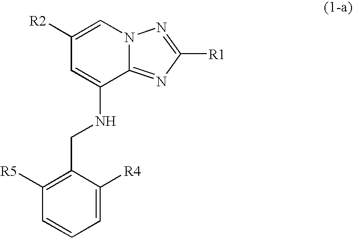

Compounds of the formula 1-a which are to be emphasized are those,

in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is halogen, fluoro-1-4C-alkyl, 2-4C-alkenyl, 2-4C-alkynyl, carboxyl, cyano, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, amino-1-4C-alkyl, mono- or di-1-4C-alkylamino-1-4C-alkyl, the radical Res-1-4C-alkyl or the radical —CO—NR31R32,
where
   Res is a imidazo, morpholino, aziridino, azetidino, pyrrolidino, pyrrolo, piperidino, piperazino or a with R30 substituted benzylamino radical and the radical Res is bonded via its nitrogen atom or one of its nitrogen atoms to the 1-4C-alkyl radical
   where
      R30 is 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen or hydroxy,
   R31 is hydrogen, hydroxyl, 1-7C-alkyl, hydroxy-1-4C-alkyl or 1-4C-alkoxy-1-4C-alkyl and
   R32 is hydrogen, 1-7C-alkyl, hydroxy-1-4C-alkyl or 1-4C-alkoxy-1-4C-alkyl,
or where
   R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, hydroxy-1-4C-alkyl, 1-4C-alkoxy, halogen or fluoro-1-4C alkyl
R5 is hydrogen, 1-4C-alkyl, hydroxy-1-4C-alkyl, 1-4C-alkoxy, halogen or fluoro-1-4C alkyl
with the proviso that R1 does not have the meaning hydrogen, 1-4C-alkyl or hydroxy-1-4C-alkyl when R2 has the meaning halogen or fluoro-1-4C-alkyl,
   and the salts of these compounds.

Particular mention may be made of those compounds of the formula 1-a,

where
R1 1-4C-alkyl, 3-7C-cycloalkyl, 3-7C-cycloalkyl-1-4C-alkyl, 1-4C-alkoxy-1-4C-alkyl, fluoro-1-4C-alkyl, hydroxy-1-4C alkyl,
R2 is carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, amino-1-4C-alkyl, mono- or di-1-4C-alkylamino-1-4C-alkyl, the radical Res-1-4C-alkyl or the radical —CO—NR31R32,
where
   Res is a imidazo, morpholino, aziridino, azetidino, pyrrolidino, pyrrolo, piperidino or piperazino radical and the radical Res is bonded via its nitrogen atom or one of its nitrogen atoms to the 1-4C-alkyl radical
   R31 is hydrogen, 1-7C-alkyl, hydroxy-1-4C-alkyl or 1-4C-alkoxy-1-4C-alkyl,
   R32 is hydrogen, 1-7C-alkyl or 1-4C-alkoxy-1-4C-alkyl,
R4 is hydrogen, 1-4C-alkyl, halogen or fluoro-1-4C-alkyl,
R5 is hydrogen, 1-4C-alkyl, halogen or fluoro-1-4C-alkyl,
   and the salts of these compounds.

Particular mention may also be made of those compounds of the formula 1-a,

where
R1 1-4C-alkyl, 3-7C-cycloalkyl, 3-7C-cycloalkyl-1-4C-alkyl, 1-4C-alkoxy-1-4C-alkyl, fluoro-1-4C-alkyl, hydroxy-1-4C alkyl,
R2 is carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, amino-1-4C-alkyl, mono- or di-1-4C-alkylamino-1-4C-alkyl, the radical Res-1-4C-alkyl or the radical —CO—NR31 R32,
where
   Res is a imidazo, morpholino, aziridino, azetidino, pyrrolidino, pyrrolo, piperidino or piperazino radical and the radical Res is bonded via its nitrogen atom or one of its nitrogen atoms to the 1-4C-alkyl radical
   R31 is hydrogen, 1-7C-alkyl, hydroxy-1-4C-alkyl or 1-4C-alkoxy-1-4C-alkyl,
   R32 is hydrogen, 1-7C-alkyl or 1-4C-alkoxy-1-4C-alkyl,
R4 is hydrogen or 1-4C-alkyl
R5 is hydrogen or 1-4C-alkyl
   and the salts of these compounds.

Compounds of the formula 1-a which are to be particularly emphasized are those,

where
R1 1-4C-alkyl
R2 is carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, amino-1-4C-alkyl, mono- or di-1-4C-alkylamino-1-4C-alkyl, the radical Res-1-4C-alkyl or the radical —CO—NR31R32,
where
   Res is a imidazo or morpholino radical and is bonded via its nitrogen atom or one of its nitrogen atoms to the 1-4C-alkyl radical
   R31 is 1-4C-alkyl or 1-4C-alkoxy-1-4C-alkyl,
   R32 is hydrogen, 1-4C-alkyl or 1-4C-alkoxy-1-4C-alkyl,
R4 is hydrogen or 1-4C-alkyl
R5 is hydrogen or 1-4C-alkyl
   and the salts of these compounds.

The compounds of the formula according to the invention can be prepared as described in an exemplary manner in the examples below, or starting from appropriate starting materials using analogous process steps or as illustrated quite generally in the scheme 1 below. Compounds of the formula 2 can be transformed to compounds of the formula 3 in a manner known per se to the person skilled in the art using standard reaction conditions, like for example using hydrogen/Pd(0). The arylation of compounds of the formula 3 to compounds of the formula 1 is carried out in manner known to the person skilled in the art using a suitable Ar—CH₂—X reactant carrying a suitable leaving group X, like for example a chlorine atom.

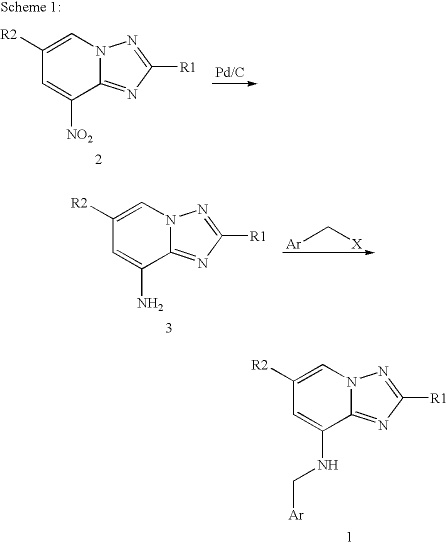

The derivatization, if any, of the compounds obtained according to the above Scheme 1 (e.g. conversion of a group R2 into another group R2) is likewise carried out in a manner known to the expert. For example, if compounds of the formula 1 where R2=carboxyl or —CO—NR31 R32 are desired, an appropriate derivatization can be performed in a manner known to the expert (for example conversion of an ester into a carboxylic acid and further transformation into an amide) at the stage of the compounds of formula 2 or 3 or more conveniently at a later point in time, for example conversion of a compound of the formula 1 into another compound of the formula 1.

If compounds of the formula 1 where R2=hydroxy-1-4C-alkyl, 1-4C-alkoxy-1-4C-alkyl, 1-4C-alkoxy-1-4C-alkoxy-1-4C-alkyl, amino-1-4C-alkyl, mono- or di-1-4C-alkylamino-1-4C-alkyl or the radical Res-1-4C-alkyl are desired, an appropriate derivatization can be performed in a manner known to the expert (for example conversion of an ester into an alcohol followed by chlorination of the alcohol and any desired substitution of the chlorine atom, like for example an etherification to form compounds of the formula 1 with R2=1-4C-alkoxy-1-4C-alkyl, 1-4C-alkoxy-1-4C-alkoxy-1-4C-alkyl or a nucleophilic substitution to form compounds of the formula 1 with R2=amino-1-4C-alkyl, mono- or di-1-4C-alkylamino-1-4C-alkyl or the radical Res-1-4C-alkyl).

Compounds of the formula 2 can be obtained in a manner known per se to the person skilled in the art, for example in analogy to the synthesis described in J. Org. Chem., 1966, 31, 260 or J. Heterocycl. Chem., 1970, 7, 1019, by cyclization of compounds of the formula 4 in the presence of a suitable carboxylic acid or a suitable ortho-ester carrying suitable substituents Z, like for example methyl groups (scheme 2).

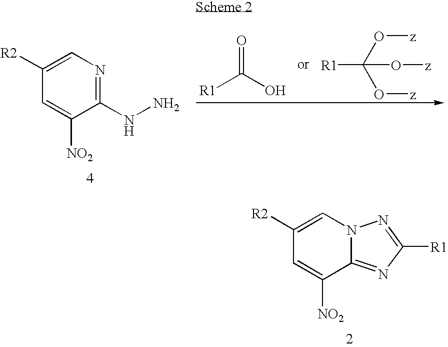

Compounds of the formula 4 are known, for example from J. Heterocycl. Chem., 1970, 7, 1019, or can be prepared in an analogous manner by reactions known per se to the person skilled in the art or in a manner as shown in a general way in scheme 3.

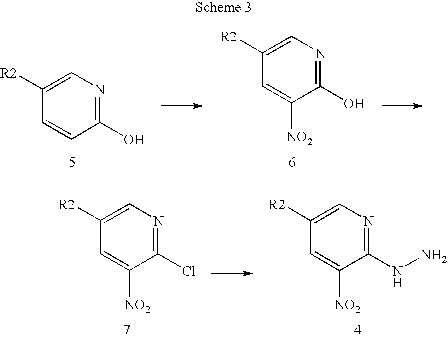

The examples below serve to Illustrate the invention in more detail without limiting it. Further compounds of the formula 1, whose preparation is not described explicitly, can likewise be prepared in an analogous manner or in a manner known per se to the person skilled in the art, using customary process techniques. The compounds named expressly as examples, and the salts of these compounds, are preferred subject matter of the invention. The abbreviation min stands for minute(s), h stands for hour(s) and m.p. stands for melting point

### EXAMPLES

### I. Final Products

### 1. 8-(2-Ethyl-6-methylbenzylamino)-2-methyl[1,2,4]triazolo[1,5-a]pyridine-6-carboxylic acid methyl ester

To a solution of 0.60 g (2.90 mmol) 2-methyl-8-amino[1,2,4]triazolo[1,5-a]pyridine-6-carboxylic acid methyl ester in DMF (12 ml) is added 3.73 g (mmol) 2-ethyl-6-methylbenzylchloride, 1.00 g (9.40 mmol), sodium carbonate and 0.10 g (0.67 mmol) sodium iodide. This reaction mixture is stirred at 25° C. for 16 h and at 55° C. for further 2.5 h. After filtration and concentration in vacuo the crude product is resolved in dichloromethane, washed with sodium hydrogen carbonate, concentrated in vacuo again and purified by column chromatography (dichloromethane/methanol: 100/1 to 100/3) to give 0.87 g (0.25 mmol/88%) of the provided product with a melting point of 139.8° C. (dichloromethane/methanol).

### 2. 8-(2-Ethyl-6-methyl-benzylamino)-2-methyl[1,2,4]triazolo[1,5-a]pyridine-6-carboxylic acid

To a suspension of 3.00 g (8.86 mmol) 8-(2-ethyl-6-methyl-benzylamino)-2-methyl[1,2,4]triazolo[1,5-a]pyridine-6-carboxylic acid methyl ester in dioxane (30 ml) is added 4.50 ml (9.00 mmol) sodium hydroxide (2N solution in water) and the mixture is stirred at 80° C. for 2.5 h. Subsequently the reaction is quenched by pouring it into an ice-cooled solution of saturated ammonium chloride. This mixture is extracted with dichloromethane and methanol (13/1) two times. The combined organic layers are concentrated in vacuo and purified by column chromatograph (dichloromethane/methanol: 13/1) to give 2.30 g (7.09 mmol/80%) of the title compound with a melting point of 264.1° C. (dichloromethane/methanol).

### 3. 8-(2-Ethyl-6-methylbenzylamino)-N,N,2-trimethyl[1,2,4]triazolo[1,5-a]pyridine-6-carboxamide

To a stirred suspension of 0.90 g (2.77 mmol) 8-(2-ethyl-6-methyl-benzylamino)-2-methyl[1,2,4]-triazolo[1,5-a]pyridine-6-carboxylic acid in a mixture of THF and DMF (5/1: 12 ml) is added 0.51 g (3.05 mmol) N,N-carbonyldiimidazole. After 1 h it is added dimethylamine (10 ml/2M solution in THF) and is stirred for further 2 h. The reaction is quenched by adding water. The organic layer is separated and the water layer is extracted with dichloromethane twice. The combined organic layers are concentrated in vacuo. The crude product is purified by column chromatography (dichloromethane/methanol: 100/3) to yield 0.45 g (1.28 mmol/46%) of the file compound.

¹H-NMR (200 MHz, d⁶-DMSO): δ=1.15 (t, 3 H), 2.34 (s, 3 H), 2.42 (s, 3 H), 2.69 (q, 2 H), 3.01 (s, 6 H), 4.41 (d, 2 H), 6.62 (s, 1 H), 7.06-7.28 (m, 3 H), 8.21 (s, 1 H).

### 4. 8-(2-Ethyl-6-methylbenzylamino)-N-(2-methoxyethyl)-2-methyl[1,2,4]triazolo-[1,5a]-pyridine-6-carboxamide

To a stirred suspension of 1.30 g (4.00 mmol) 8-(2-ethyl-6-methyl-benzylamino)-2-methyl[1,2,4]triazolo[1,5-a]pyridine-6-carboxylic acid in a mixture of THF and DMF (5/1: 18 ml) is added 0.51 g (3.05 mmol) N,N-carbonyldiimidazole. After 1 h it is added 2-methoxyethylamin and is stirred for further 2 h. The reaction is quenched by adding water. The organic layer is separated and the water layer is extracted with dichloromethane twice. The combined organic layers are concentrated in vacuo. The crude product is purified by column chromatography (dichloromethane/methanol: 100/3) to yield 0.70 g (1.83 mmol/46%) of the title compound with a melting point of 135-136° C. (dichloromethane).

### 5. 8-(2-Ethyl-6-methylbenzylamino)-6-methoxymethyl-2-methyl[1,2,4]triazolo-[1,5-a]pyridine

A solution of 0.80 g (2.19 mmol) 8-(2-ethyl-6-methylbenzylamino)-6-chloromethyl-2-methyl[1,2,4]triazolo[1,5-a]pyridine hydrochloride and sodium methoxide (4.00 ml/30% solution in methanol) in methanol (6.00 ml) is stirred in the micro wave reactor at 100° C. for 15 min. The reaction is quenched by pouring it into a saturated ammonium chloride solution. This mixture is extracted with dichloromethane three times. The combined organic layers are concentrated in vacuo and the crude product is purified by column chromatography (diethyl ether/diisopropyl ether: 1/1) to give 0.45 g (1.38 mmol/63%) of the title compound with a melting point of 98.7° C. (diethyl ether/diisopropyl ether).

### 6. 8-(2-Ethyl-6-methylbenzylamino)-6-hydroxymethyl-2-methyl[1,2,4]triazolo-[1,5-a]pyridine

To a solution of 9.00 g (26.0 mmol) 8-(2-ethyl-6-methyl-benzylamino)-2-methyl[1,2,4]triazolo-[1,5-a]pyridine-6-carboxylic acid methyl ester in THF (180 ml) is added 1.20 g (32.0 mmol) lithium aluminium hydride and is stirred for at 25° C. for 1 h. The reaction is quenched by adding slowly water (2 ml) and sodium hydroxide solution (6N/2 ml). The inorganic solid is filtered off and washed with a mixture of dichloromethane and methanol (13/1). The combined filtrate is concentrated in vacuo and the crude product is purified by column chromatography (dichloromethane/methanol: 13/1) to yield 8.20 g (26.4 mol/99%) of the title compound with a melting point of 180.3° C. (dichloromethane/methanol).

### 7. 8-(2-Ethyl-6-methylbenzylamino)-6-dimethylaminomethyl-2-methyl[1,2,4]triazolo[1,5-a]pyridine hydrochloride

To a suspension of 1.20 g (3.29 mmol) 8-(2-ethyl-6-methylbenzylamino)-6-chloromethyl-2-methyl[1,2,4]triazolo[1,5-a]pyridine hydrochloride in THF (10 ml) is added dimethylamine (20 ml/2M solution in THF) and is stirred at 25° C. for 18 h. The reaction mixture is concentrated in vacuo and resolved in dichloromethane. After washing with saturated hydrogen carbonate solution the organic layer is concentrated in vacuo and the crude product is purified by column chromatography (diethyl ether/triethylamine: 95/5). Afterwards to the in diethyl ether dissolved product is added hydrochloric acid (5N solution in diethyl ether) to give 0.80 g (2.14 mmol/65%) of the title compound with a melting point of 235.8° C. (diethyl ether).

### 8. 8-(2-Ethyl-6-methylbenzylamino)-6-methylaminomethyl-2-methyl[1,2,4]triazolo-[1,5-a]-pyridine

To a suspension of 1.00 g (2.74 mmol) 8-(2-ethyl-6-methylbenzylamino)-6-chloromethyl-2-methyl[1,2,4]triazolo[1,5-a]pyridine hydrochloride in THF (5.0 ml) is added methylamine (35 ml/2M solution in THF) and is stirred at 25° C. for 72 h. The reaction mixture is concentrated in vacuo and resolved in dichloromethane. After washing with saturated hydrogen carbonate solution the organic layer is concentrated in vacuo and the crude product is purified by column chromatography (dichloromethane/methanol: 100/3 to 13/1) to give 0.34 g (1.05 mmol/38%) of the title product with a melting point of 179.0° C. (dichloromethane/methanol).

### 9. 8-(2-Ethyl-6-methylbenzylamino)-6-aminomethyl-2-methyl[1,2,4]triazolo-[1,5-a]pyridine

A mixture of 1.00 g (2.74 mmol) 8-(2-ethyl-6-methylbenzylamino)-6-chloromethyl-2-methyl[1,2,4]triazolo[1,5-a]pyridine hydrochloride and ammonia (10 ml/2M solution in methanol) is stirred at 80° C. for 2 h in a micro wave reactor. Subsequently the reaction mixture is concentrated in vacuo and resolved in dichloromethane. After washing with saturated hydrogen carbonate solution the organic layer is concentrated in vacuo and the crude product is purified by column chromatography (dichloromethane/methanol: 100/3 to 13/1) to give 0.17 g (0.55 mmol/20%) of the title product.

¹H-NMR (200 MHz, CDCl₃): δ=1.15 (t, 3 H), 2.34 (s, 3 H), 2.36 (s, 3 H); 2.68 (q, 2 H), 3.74 (s, 1 H), 4.37 (d, 2 H), 6.69 (s, 1 H), 7.07-7.24 (m, 3 H), 8.00 (s, 1 H).

### 10. 8-(2-Ethyl-6-methylbenzylamino)-6-morpholinomethyl-2-methyl[1,2,4]triazolo-[1,5-a]-pyridine

A suspension of 1.00 g (2.74 mmol) 8-(2-ethyl-6-methylbenzylamino)-6-chloromethyl-2-methyl[1,2,4]triazolo[1,5-a]pyridine hydrochloride and 0.95 g (11.0 mmol) morpholine in THF (8 ml) is stirred at 100° C. for 30 min in a micro wave reactor. Subsequently the reaction mixture is concentrated in vacuo and the crude product is purified by column chromatography (dichloromethane/methanol: 100/1 to 100/3). The product is reslurried in diisopropyl ether and dried in vacuo to give 0.48 g (1.26 mmol/48%) of the title product with a melting point of 97.2° C. (diisopropyl ether).

### 11. 8-(2-Ethyl-6-methylbenzylamino)-6-(imidazol-1-ylmethyl)-2-methyl[1,2,4]triazolo-[1,5-a]-pyridine

A suspension of 1.00 g (2.74 mmol) 8-(2-ethylmethylbenzylamino)-6-chloromethyl-2-methyl[1,2,4]triazolo[1,5-a]pyridine hydrochloride and 1.00 g (14.5 mmol) imidazole in THF (10 ml) is stirred at 120° C. for 3 h in a micro wave reactor. Subsequently the reaction mixture is concentrated in vacuo and resolved in dichloromethane. After washing with saturated hydrogen carbonate solution the organic layer is concentrated in vacuo and the crude product is purified by column chromatography (dichloromethane/methanol: 100/3 to 13/1) to give 0.30 g (0.83 mmol/30%) of the title product with a melting point of 158.5° C. (dichloromethane/methanol).

### 12. 8-(2-Ethyl-6-methylbenzylamino)-6-(2-methoxyethoxymethyl)-2-methyl-[1,2,4]triazolo[1,5-a]pyridine

To 2-methoxyethanol (10 ml) is added 1.00 g (43.5 mmol) sodium and it is stirred at 25° C. until the hydrogen release stops. Subsequently 1.00 g (2.74 mmol) 8-(2-ethyl-6-methylbenzylamino)-6-chloromethyl-2-methyl[1,2,4]triazolo[1,5-a]pyridine hydrochloride is added and the mixture is stirred for further 18 h. The reaction is quenched by pouring it into a saturated ammonium chloride solution and is extracted with dichloromethane two times. The combined organic layers are concentrated in vacuo and the crude product is purified by column chromatography (dichloromethane/methanol: 100/1 to 13/1) to give 0.78 g (2.12 mmol/77%) of the title product.

¹H-NMR (200 MHz, d⁶-DMSO): δ=1.16 (t, 3 H), 2.34 (s, 3 H), 2.39 (s, 3 H); 2.69 (q, 2 H), 3.28 (s, 3 H), 3.58-3.63 (m, 4 H), 4.39 (d, 2 H), 4.51 (s, 2 H), 6.60 (s, 1 H), 7.06-7.23 (m, 3 H), 8.05 (s, 1 H).

### 13. 8-(2-Ethyl-6-methylbenzylamino)-6-(2-methoxyethoxymethyl)-2-methyl-[1,2,4]triazolo[1,5-a]pyridine hydrochloride

To a solution of 0.78 g (2.12 mmol) 8-(2-ethyl-6-methylbenzylamino)-6-(2-methoxyethoxy-methyl)-2-methyl-[1,2,4]triazolo[1,5-a]pyridine in dichloromethane (10 ml) is added hydrochloric acid (5N solution in diethyl ether). The reaction mixture is concentrated in vacuo and the crude product is crystallised in diethyl ether to give 0.60 g (1.48 mmol/54%) of the title compound with a melting point of 120.2° C. (diethyl ether).

### II. Starting Compounds and Intermediates

### A. 6-Chloro-5-nitro-nicotinic acid methyl ester

To a suspension of 49.0 g (0.27 mol) 6-hydroxy-5-nitro-nicotinic acid in thionyl chloride (240 ml) is added DMF (2 ml). This mixture is stirred at 60° C. and after gassing stops it is stirred at 80° C. for further 18 h. The thionyl chloride is removed under vacuo and the residue is coevaporated with toluene three times. Subsequently this reaction mixture is dissolved in dichloromethane (100 ml) and cooled to 0° C. before methanol (55.5 ml) is dropwise added. The precipitated solid is filtered off and dried under vacuo at 50° C. to give 27.6 g (13.7 mmol/52%) of the titled compound as a light yellow solid with a melting point of 78° C. (dichloromethane/methanol).

### B. 6-Hydrazino-5-nitro-nicotinic acid methyl ester

To a at 15° C. cooled solution of 30.0 g (0.14 mol) 6-chloro-5-nitro-nicotinic acid methyl ester in dioxane (600 ml) is added hydrazine hydrate (21.5 ml). During the addition the reaction mixture is warmed up to 25° C. and is stirred for further 3 h. The reaction is quenched by pouring the reaction mixture into a saturated aqueous ammonium chloride solution. The precipitated solid is filtered off and dried under vacuo at 50° C. to give 26.5 g (0.12 mol/90%) of the title compound as a red solid.

¹H-NMR (200 MHz, d⁶-DMSO): δ=3.85 (s, 3 H), 8.69 (d, 1 H), 8.90 (d, 1 H).

### C. 2-Methyl-8-nitro[1,2,4]triazolo[1,5-a]pyridine-6-carboxylic acid methyl ester

A suspension of 20.0 g (94.0 mmol) 6-hydrazino-5-nitro-nicotinic acid methyl ester in acetic acid (108 ml) is stirred at reflux for 24 h. Afterwards the reaction mixture is concentrated in vacuo, coevaporated with toluene twice, resolved in water and neutralised with sodium hydrogen carbonate. The precipitated solid is filtered off, washed with water, dried under vacuo at 60° C. and purified by column chromatography (dichloromethane/methanol: 100/1 to 100/3) to provide 15.3 g (64.9 mmol/69%) of the title compound.

¹H-NMR (200 MHz, d6-DMSO): δ=2.62 (s, 3 H), 3.97 (s, 3 H), 8.80 (d, 1 H), 9.81 (d, 1 H).

### D. 2-Methyl-8-amino[1,2,4]triazolo[1,5-a]pyridine-6-carboxylic acid methyl ester

A suspension of 3.60 g (15.2 mmol) 2-methyl-8-nitro[1,2,4]triazolo[1,5-a]pyridine-6-carboxylic acid methyl ester, 1.00 g (0.94 mmol) palladium on carbon (10% on carbon) and 15.0 ml (0.16 mol) cyclohexadiene in ethyl acetate (75 ml) is stirred at reflux for 4 h. Subsequently the catalyst is filtered off and the reaction mixture is concentrated in vacuo. The crude product is reslurried in acetone and dried in vacuo to give 0.65 g (3.15 mmol/21%) of the titled product with a melting point of 212.2° C. (acetone).

### E. 8-(2-Ethyl-6-methylbenzylamino)-6-chloromethyl-2-methyl[1,2,4]triazolo-[1,5-a]pyridine hydrochloride

To a stirred solution of 1.00 g (3.20 mmol) 8-(2-ethyl-6-methylbenzylamino)-6-hydroxymethyl-2-methyl[1,2,4]triazolo[1,5-a]pyridine in dichloromethane (20 ml) is added 2.00 ml (27.41 mmol) thionyl chloride and it is stirred for further 0.5 h. Subsequently to the reaction mixture is added toluene and is coevaporated two times to give 1.30 g (3.20 mmol/100%) of the crude product as an ochre-brown solid with a melting point of 197.8° C. This product is used without any purification and further characterisation for the next (alkylation reaction) transformation.

### Commercial Utility

The compounds of the formula 1 and their salts have valuable pharmacological properties which make them commercially utilizable. In particular, they exhibit marked inhibition of gastric acid secretion and an excellent gastric and intestinal protective action in warm-blooded animals, in particular humans. In this connection, the compounds according to the invention are distinguished by a high selectivity of action, an advantageous duration of action, a particularly good enteral activity, the absence of significant side effects and a large therapeutic range.

“Gastric and intestinal protection” in this connection is understood as meaning the prevention and treatment of gastrointestinal diseases, in particular of gastrointestinal inflammatory diseases and lesions (such as, for example, gastric ulcer, peptic ulcer, including peptic ulcer bleeding, duodenal ulcer, gastritis, hyperacidic or medicament-related functional dyspepsia), which can be caused, for example, by microorganisms (e.g. Helicobacter pylori), bacterial toxins, medicaments (e.g. certain antiinflammatories and antirheumatics, such as NSAIDs and COX-inhibitors), chemicals (e.g. ethanol), gastric acid or stress situations. “Gastric and intestinal protection” is understood to include, according to general knowledge, gastroesophageal reflux disease (GERD), the symptoms of which include, but are not limited to, heartburn and/or acid regurgitation.

In their excellent properties, the compounds according to the invention surprisingly prove to be clearly superior to the compounds known from the prior art in various models in which the antiulcerogenic and the antisecretory properties are determined. On account of these properties, the compounds of the formula 1 and their pharmacologically acceptable salts are outstandingly suitable for use in human and veterinary medicine, where they are used, in particular, for the treatment and/or prophylaxis of disorders of the stomach and/or intestine.

A further subject of the invention are therefore the compounds according to the invention for use in the treatment and/or prophylaxis of the abovementioned diseases.

The invention likewise includes the use of the compounds according to the invention for the production of medicaments which are employed for the treatment and/or prophylaxis of the abovementioned diseases.

The invention furthermore includes the use of the compounds according to the invention for the treatment and/or prophylaxis of the abovementioned diseases.

A further subject of the invention are medicaments which comprise one or more compounds of the formula 1 and/or their pharmacologically acceptable salts.

The medicaments are prepared by processes which are known per se and familiar to the person skilled in the art. As medicaments, the pharmacologically active compounds according to the invention (=active compounds) are either employed as such, or preferably in combination with suitable pharmaceutical auxiliaries or excipients in the form of tablets, coated tablets, capsules, suppositories, patches (e.g. as TTS), emulsions, suspensions or solutions, the active compound content advantageously being between 0.1 and 95% and it being possible to obtain a pharmaceutical administration form exactly adapted to the active compound and/or to the desired onset and/or duration of action (e.g. a sustained-release form or an enteric form) by means of the appropriate selection of the auxiliaries and excipients.

The auxiliaries and excipients which are suitable for the desired pharmaceutical formulations are known to the person skilled in the art on the basis of his/her expert knowledge. In addition to solvents, gel-forming agents, suppository bases, tablet auxiliaries and other active compound excipients, it is possible to use, for example, antioxidants, dispersants, emulsifiers, antifoams, flavor corrigents, preservatives, solubilizers, colorants or, in particular, permeation promoters and complexing agents (e.g. cyclodextrins).

The active compounds can be administered orally, parenterally or percutaneously. In general, it has proven advantageous in human medicine to administer the active compound(s) in the case of oral administration in a daily dose of approximately 0.01 to approximately 20, preferably 0.05 to 5, in particular 0.1 to 1.5, mg/kg of body weight, if appropriate in the form of several, preferably 1 to 4, individual doses to achieve the desired result. In the case of a parenteral treatment, similar or (in particular in the case of the intravenous administration of the active compounds), as a rule, lower doses can be used. The establishment of the optimal dose and manner of administration of the active compounds necessary in each case can easily be carried out by any person skilled in the art on the basis of his/her expert knowledge.

If the compounds according to the invention and/or their salts are to be used for the treatment of the abovementioned diseases, the pharmaceutical preparations can also contain one or more pharmacologically active constituents of other groups of medicaments, for example: tranquilizers (for example from the group of the benzodiazepines, for example diazepam), spasmolytics (for example, bietamiverine or camylofine), anticholinergics (for example, oxyphencyclimine or phencarbamide), local anesthetics, (for example, tetracaine or procaine), and, if appropriate, also enzymes, vitamins or amino acids.

To be emphasized in this connection is in particular the combination of the compounds according to the invention with pharmaceuticals which inhibit acid secretion, such as, for example, H₂ blockers (e.g. cimetidine, ranitidine), H⁺/K⁺ ATPase inhibitors (e.g. omeprazole, pantoprazole), or further with so-called peripheral anticholinergics (e.g. pirenzepine, telenzepine) and with gastrin antagonists with the aim of increasing the principal action in an additive or super-additive sense and/or of eliminating or of decreasing the side effects, or further the combination with antibacterially active substances (such as, for example, cephalosporins, tetracyclines, penicillins, macrolides, nitroimidazoles or alternatively bismuth salts) for the control of Helicobacter pylori. Suitable antibacterial co-components which may be mentioned are, for example, meziocillin, ampicillin, amoxicillin, cefalothin, cefoxitin, cefotaxime, imipenem, gentamycin, amikacin, erythromycin, dprofloxacin, metronidazole, clarithromycin, azithromycin and combinations thereof (for example clarithromycin+metronidazole).

In view of their excellent gastric and intestinal protection action, the compounds of formula 1 are suited for a free or fixed combination with those medicaments (e.g. certain antinflammatories and antirheumatics, such as NSAIDs), which are known to have a certain ulcerogenic potency. In addition, the compounds of formula 1 are suited for a free or fixed combination with motility-modifying drugs.

### Pharmacology

The excellent gastric protective action and the gastric acid secretion-inhibiting action of the compounds according to the invention can be demonstrated in investigations on animal experimental models. The compounds according to the invention investigated in the model mentioned below have been provided with numbers which correspond to the numbers of these compounds in the examples.

### Testing of the Secretion-Inhibiting Action on the Perfused Rat Stomach

In Table A which follows, the influence of the compounds according to the invention on the pentagastrin-stimulated acid secretion of the perfused rat stomach after intraduodenal administration in vivo is shown.

TABLE A

| | Dose | |
|---|---|---|
| | (μmol/kg) | Inhibition of acid secretion |
| No. | i.d. | (%) |
| 3 | 1 | >25 |
| 4 | 1 | <25 |
| 5 | 1 | >25 |
| 6 | 1 | <25 |
| 7 | 1 | >25 |
| 8 | 1 | >25 |
| 9 | 1 | <25 |
| 10 | 1 | <25 |
| 11 | 1 | >25 |
| 13 | 1 | >25 |

### Methodology

The abdomen of anesthetized rats (CD rat, female, 200-250 g; 1.5 g/kg i.m. urethane) was opened after tracheotomy by a median upper abdominal incision and a PVC catheter was fixed transorally in the esophagus and another via the pylorus such that the ends of the tubes just projected into the gastric lumen. The catheter leading from the pylorus led outward into the right abdominal wall through a side opening.

After thorough rinsing (about 50-100 ml), warm (37° C.) physiological NaCl solution was continuously passed through the stomach (0.5 m/min, pH 6.8-6.9; Braun-Unita I). The pH (pH meter 632, glass electrode EA 147; φ=5 mm, Metrohm) and, by titration with a freshly prepared 0.01N NaOH solution to pH 7 (Dosimat 665 Metrohm), the secreted HCl were determined in the effluent in each case collected at an interval of 15 minutes.

The gastric secretion was stimulated by continuous infusion of 1 μg/kg (=1.65 ml/h) of i.v. pentagastrin (left femoral vein) about 30 min after the end of the operation (i.e. after determination of 2 preliminary fractions). The substances to be tested were administered intraduodenally in a 2.5 ml/kg liquid volume 60 min after the start of the continuous pentagastrin infusion.

The body temperature of the animals was kept at a constant 37.8-38° C. by infrared irradiation and heat pads (automatic, stepless control by means of a rectal temperature sensor).

1. A compound of the formula 1 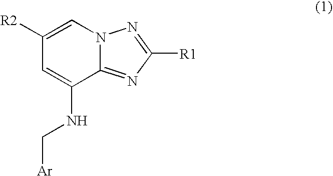
in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is halogen, fluoro-1-4C-alkyl, 2-4C-alkenyl, 2-4C-alkynyl, carboxyl, cyano, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, amino-1-4C-alkyl, mono- or di-1-4C-alkylamino-1-4C-alkyl, the radical Res-1-4C-alkyl or the radical —CO—NR31R32,
where
Res is a imidazo, morpholino, aziridino, azetidino, pyrrolidino, pyrrolo, piperidino, piperazino or a with R30 substituted benzylamino radical and the radical Res is bonded via its nitrogen atom or one of its nitrogen atoms to the 1-4C-alkyl radical,
where
R30 is 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen or hydroxy,
R31 is hydrogen, hydroxyl, 1-7C-alkyl, hydroxy-1-4C-alkyl or 1-4C-alkoxy-1-4C-alkyl and
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, piperidino, piperazino, N-1-4C-alkylpiperazino, morpholino, aziridino or azetidino group,
Ar is a R4, R5, R6 and R7 substituted mono- or bicyclic aromatic residue selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
wherein
R4 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, fluoro-1-4C-alkyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R5 is hydrogen, 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen, hydroxy, aryl, aryl-1-4C-alkyl, aryl-oxy, aryl-1-4C-alkoxy, fluoro-1-4C-alkyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R6 is hydrogen, 1-4C-alkyl or halogen and
R7 is hydrogen, 1-4C-alkyl or halogen,
wherein
aryl is phenyl or substituted phenyl with one, two or three same or different substituents selected from the group consisting of 1-4C-alkyl, 1-4C-alkoxy, carboxy, 1-4C-alkoxycarbonyl, halogen, trifluoromethyl, nitro, trifluoromethoxy, hydroxy and cyano,
with the proviso that R1 does not have the meaning hydrogen, 1-4C-alkyl or hydroxy-1-4C-alkyl when R2 has the meaning halogen or fluoro-1-4C-alkyl,
or a salt thereof.

2. A compound of the formula 1 as claimed in claim 1,
in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is halogen, fluoro-1-4C-alkyl, 2-4C-alkenyl, 2-4C-alkynyl, carboxyl, cyano, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, amino-1-4C-alkyl, mono- or di-1-4C-alkylamino-1-4C-alkyl, the radical Res-1-4C-alkyl or the radical —CO—NR31R32,
where
Res is a imidazo, morpholino, aziridino, azetidino, pyrrolidino, pyrrolo, piperidino, piperazino or a with R30 substituted benzylamino radical and the radical Res is bonded via its nitrogen atom or one of its nitrogen atoms to the 1-4C-alkyl radical,
where
R30 is 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen or hydroxy,
R31 is hydrogen, hydroxyl, 1-7C-alkyl, hydroxy-1-4C-alkyl or 1-4C-alkoxy-1-4C-alkyl and
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, piperidino, piperazino, N-1-4C-alkylpiperazino, morpholino, aziridino or azetidino group,
Ar is a R4, R5, R6 and R7 substituted mono- or bicyclic aromatic residue selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
wherein
R4 is hydrogen, 1-4C-alkyl, hydroxy-1-4C-alkyl, 1-4C-alkoxy, halogen or fluoro-1-4C alkyl,
R5 is hydrogen, 1-4C-alkyl, hydroxy-1-4C-alkyl, 1-4C-alkoxy, halogen or fluoro-1-4C alkyl,
R6 is hydrogen,
R7 is hydrogen,
with the proviso that R1 does not have the meaning hydrogen, 1-4C-alkyl or hydroxy-1-4C-alkyl when R2 has the meaning halogen or fluoro-1-4C-alkyl,
or a salt thereof.

3. A compound of the formula 1 as claimed in claim 1,
in which
R1 is 1-4C-alkyl, 3-7C-cycloalkyl, 3-7C-cycloalkyl-1-4C-alkyl, 1-4C-alkoxy-1-4C-alkyl, fluoro-1-4C-alkyl or hydroxy-1-4C alkyl,
R2 is carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, amino-1-4C-alkyl, mono- or di-1-4C-alkylamino-1-4C-alkyl, the radical Res-1-4C-alkyl or the radical —CO—NR31R32,
where
Res is a imidazo, morpholino, aziridino, azetidino, pyrrolidino, pyrrolo, piperidino or piperazino radical and the radical Res is bonded via its nitrogen atom or one of its nitrogen atoms to the 1-4C-alkyl radical,
R31 is hydrogen, 1-7C-alkyl, hydroxy-1-4C-alkyl or 1-4C-alkoxy-1-4C-alkyl,
R32 is hydrogen, 1-7C-alkyl or 1-4C-alkoxy-1-4C-alkyl,
Ar is a R4, R5, R6 and R7 substituted monocyclic aromatic residue selected from the group consisting of phenyl, pyridinyl, thiophenyl, furanyl and pyrrolyl,
wherein
R4 is hydrogen, 1-4C-alkyl, halogen or fluoro-1-4C-alkyl,
R5 is hydrogen, 1-4C-alkyl, halogen or fluoro-1-4C-alkyl,
R6 is hydrogen,
R7 is hydrogen,
or a salt thereof.

4. A compound of the formula 1 as claimed in claim 1,
in which
R1 is 1-4C-alkyl, 3-7C-cycloalkyl, 3-7C-cycloalkyl-1-4C-alkyl, 1-4C-alkoxy-1-4C-alkyl, fluoro-1-4C-alkyl or hydroxy-1-4C alkyl,
R2 is carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, amino-1-4C-alkyl, mono- or di-1-4C-alkylamino-1-4C-alkyl, the radical Res-1-4C-alkyl or the radical —CO—NR31R32,
where
Res is a imidazo, morpholino, aziridino, azetidino, pyrrolidino, pyrrolo, piperidino or piperazino radical and the radical Res is bonded via its nitrogen atom or one of its nitrogen atoms to the 1-4C-alkyl radical,
R31 is hydrogen, 1-7C-alkyl, hydroxy-1-4C-alkyl or 1-4C-alkoxy-1-4C-alkyl,
R32 is hydrogen, 1-7C-alkyl or 1-4C-alkoxy-1-4C-alkyl,
Ar is R4, R5, R6 and R7 substituted phenyl,
wherein
R4 is hydrogen or 1-4C-alkyl,
R5 is hydrogen or 1-4C-alkyl,
R6 is hydrogen,
R7 is hydrogen,
or a salt thereof.

5. A compound of the formula 1 as claimed in claim 1,
in which
R1 is 1-4C-alkyl,
R2 is carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, amino-1-4C-alkyl, mono- or di-1-4C-alkylamino-1-4C-alkyl, the radical Res-1-4C-alkyl or the radical —CO—NR31R32,
where
Res is a imidazo or morpholino radical and is bonded via its nitrogen atom or one of its nitrogen atoms to the 1-4C-alkyl radical,
R31 is 1-4C-alkyl or 1-4C-alkoxy-1-4C-alkyl,
R32 is hydrogen, 1-4C-alkyl or 1-4C-alkoxy-1-4C-alkyl,
Ar is R4, R5, R6 and R7 substituted phenyl,
wherein
R4 is hydrogen or 1-4C-alkyl,
R5 is hydrogen or 1-4C-alkyl,
R6 is hydrogen,
R7 is hydrogen,
or a salt thereof.

6. A compound of the formula 1 as claimed in claim 1, characterized by the formula 1-a, 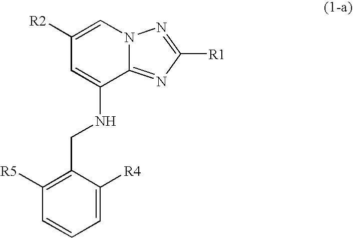
in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is halogen, fluoro-1-4C-alkyl, 2-4C-alkenyl, 2-4C-alkynyl, carboxyl, cyano, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl, amino-1-4C-alkyl, mono- or di-1-4C-alkylamino-1-4C-alkyl, the radical Res-1-4C-alkyl or the radical —CO—NR31R32,
where
Res is a imidazo, morpholino, aziridino, azetidino, pyrrolidino, pyrrolo, piperidino, piperazino or a with R30 substituted benzylamino radical and the radical Res is bonded via its nitrogen atom or one of its nitrogen atoms to the 1-4C-alkyl radical,
where
R30 is 1-4C-alkyl, hydroxy-1-4C-alkyl, 1-4C-alkoxy, 2-4C-alkenyloxy, 1-4C-alkylcarbonyl, carboxy, 1-4C-alkoxycarbonyl, carboxy-1-4C-alkyl, 1-4C-alkoxycarbonyl-1-4C-alkyl, halogen or hydroxy,
R31 is hydrogen, hydroxyl, 1-7C-alkyl, hydroxy-1-4C-alkyl or 1-4C-alkoxy-1-4C-alkyl and
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, piperidino, piperazino, N-1-4C-alkylpiperazino, morpholino, aziridino or azetidino group,
R4 is hydrogen, 1-4C-alkyl, hydroxy-1-4C-alkyl, 1-4C-alkoxy, halogen or fluoro-1-4C alkyl,
R5 is hydrogen, 1-4C-alkyl, hydroxy-1-4C-alkyl, 1-4C-alkoxy, halogen or fluoro-1-4C alkyl,
with the proviso that R1 does not have the meaning hydrogen, 1-4C-alkyl or hydroxy-1-4C-alkyl when R2 has the meaning halogen or fluoro-1-4C-alkyl,
or a salt thereof.

7. A compound of the formula 1-a as claimed in claim 6,
in which
R1 is 1-4C-alkyl, 3-7C-cycloalkyl, 3-7C-cycloalkyl-1-4C-alkyl, 1-4C-alkoxy-1-4C-alkyl, fluoro-1-4C-alkyl or hydroxy-1-4C alkyl,
R2 is carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, amino-1-4C-alkyl, mono- or di-1-4C-alkylamino-1-4C-alkyl, the radical Res-1-4C-alkyl or the radical —CO—NR31R32,
where
Res is a imidazo, morpholino, aziridino, azetidino, pyrrolidino, pyrrolo, piperidino or piperazino radical and the radical Res is bonded via its nitrogen atom or one of its nitrogen atoms to the 1-4C-alkyl radical,
R31 is hydrogen, 1-7C-alkyl, hydroxy-1-4C-alkyl or 1-4C-alkoxy-1-4C-alkyl,
R32 is hydrogen, 1-7C-alkyl or 1-4C-alkoxy-1-4C-alkyl,
R4 is hydrogen, 1-4C-alkyl, halogen or fluoro-1-4C-alkyl,
R5 is hydrogen, 1-4C-alkyl, halogen or fluoro-1-4C-alkyl,
or a salt thereof.

8. A compound of the formula 1-a as claimed in claim 6,
in which
R1 is 1-4C-alkyl, 3-7C-cycloalkyl, 3-7C-cycloalkyl-1-4C-alkyl, 1-4C-alkoxy-1-4C-alkyl, fluoro-1-4C-alkyl or hydroxy-1-4C alkyl,
R2 is carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, amino-1-4C-alkyl, mono- or di-1-4C-alkylamino-1-4C-alkyl, the radical Res-1-4C-alkyl or the radical —CO—NR31R32,
where
Res is a imidazo, morpholino, aziridino, azetidino, pyrrolidino, pyrrolo, piperidino or piperazino radical and the radical Res is bonded via its nitrogen atom or one of its nitrogen atoms to the 1-4C-alkyl radical,
R31 is hydrogen, 1-7C-alkyl, hydroxy-1-4C-alkyl or 1-4C-alkoxy-1-4C-alkyl,
R32 is hydrogen, 1-7C-alkyl or 1-4C-alkoxy-1-4C-alkyl,
R4 is hydrogen or 1-4C-alkyl,
R5 is hydrogen or 1-4C-alkyl,
or a salt thereof.

9. A compound of the formula 1-a as claimed in claim 6,
in which
R1 is 1-4C-alkyl,
R2 is carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, amino-1-4C-alkyl, mono- or di-1-4C-alkylamino-1-4C-alkyl, the radical Res-1-4C-alkyl or the radical —CO—NR31R32,
where
Res is a imidazo or morpholino radical and is bonded via its nitrogen atom or one of its nitrogen atoms to the 1-4C-alkyl radical,
R31 is 1-4C-alkyl or 1-4C-alkoxy-1-4C-alkyl,
R32 is hydrogen, 1-4C-alkyl or 1-4C-alkoxy-1-4C-alkyl,
R4 is hydrogen or 1-4C-alkyl,
R5 is hydrogen or 1-4C-alkyl,
or a salt thereof.

10. A pharmaceutical composition comprising a compound of formula 1 as claimed in claim 1 and/or a pharmacologically acceptable salt thereof, together with a pharmaceutically acceptable auxiliary and/or excipient.

11. A method of treating a gastrointestinal disorder in a patient comprising administering to a patient in need thereof a compound of formula 1 as claimed in claim 1 or a pharmacologically acceptable salt thereof.
